(19) United States
(12) Patent Application Publication
(10) Pub. No.: US 2007/0009472 A1
(43) Pub. Date: Jan. 11, 2007

(54) Personal care compositions comprising a non-binding thickener with a metal ion

(76) Inventors: Michael Frederick Niebauer, Cincinnati, OH (US); Brandon Scott Lane, Hamilton, OH (US); James Robert Schwartz, West Chester, OH (US); David Thomas Warnke, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/479,765

(22) Filed: Jun. 30, 2006

Related U.S. Application Data
(60) Provisional application No. 60/696,928, filed on Jul. 6, 2005.

(51) Int. Cl. A61K 33/32

(52) U.S. Cl. 424/70.28; 424/642

(57) ABSTRACT

Personal care composition comprising an effective amount of a zinc containing material; a surfactant; a cationic, or a nonionic thickening polymer, or a mixture thereof. The present invention may be a multi-phase personal care composition comprising: at least two phases wherein at least one of the phases comprises an effective amount of a zinc containing material, at least one surfactant, and at least one cationic or nonionic thickening polymer, or a mixture thereof, and the at least two phases are visually distinct phases that are packaged in physical contact and maintain stability. In the present invention, the at least one cationic or nonionic thickening polymers, or mixtures thereof, does not bind completely to the zinc ions from the zinc containing material.

### CROSS REFERENCE TO RELATED APPLICATION

This application claims the benefit of U.S. Provisional Application No. 60/696,928 filed Jul. 6, 2006.

### FIELD

Certain embodiments of the present invention relate to personal care compositions and methods of treating microbial and fungal infections on the skin or scalp. Additionally, certain embodiments of the present invention relate to methods for the treatment of dandruff and compositions which provide improved anti-dandruff activity.

### BACKGROUND

Of the trace metals, zinc is the second most abundant metal in the human body, catalyzing nearly every bio-process directly or indirectly through inclusion in many different metalloenzymes. The critical role zinc plays can be discerned from the symptoms of dietary deficiency, which include dermatitis, anorexia, alopecia and impaired overall growth. Zinc appears especially important to skin health and has been used (typically in the form of zinc oxide or calamine) for over 3000 years to control a variety of skin problems.

Inorganic salts, such as zinc oxide, have been employed as bacteriostatic and/or fungistatic compounds in a large variety of products including paints, coatings, and antiseptics; However, zinc salts do not possess as high of a level of biocidal efficacy as might be desired for many anti-dandruff and skin care applications.

The Theological properties of a personal care composition impact the consumer acceptance. In order to move to higher rheologies it is important to find a thickener that does not bind completely with a metal ion. Out of the three classes of polymeric thickeners; cationic, anionic, and nonionic, the anionic thickeners tend to bind more with the metal ions producing a consumer unacceptable composition. Cationic and nonionic thickeners can raise the rheology profile of these systems without reducing the efficiency of the formula.

### SUMMARY

The present invention is related to a personal care composition comprising an effective amount of a zinc containing material; a surfactant; a cationic, or a nonionic thickening polymer, or a mixture thereof.

The present invention may be a multi-phase personal care composition comprising: at least two phases wherein at least one of the phases comprises an effective amount of a zinc containing material, at least one surfactant, and at least one cationic or nonionic thickening polymer, or a mixture thereof, and the at least two phases are visually distinct phases that are packaged in physical contact and maintain stability.

In the present invention, the at least one cationic or nonionic thickening polymers, or mixtures thereof, does not bind completely to the zinc ions from the zinc containing material.

These and other features, aspects, and advantages of the present invention will become evident to those skilled in the art from a reading of the present disclosure.

### DETAILED DESCRIPTION

The essential components of the personal care composition are described below. Also included is a nonexclusive description of various optional and preferred embodiments useful in embodiments of the present invention. While the specification concludes with claims which particularly point out and distinctly claim the invention, it is believed the present invention will be better understood from the following description.

All percentages, parts, and ratios are based upon the total weight of the compositions of the present invention, unless otherwise specified. All such weights as they pertain to listed ingredients are based on the active level and, therefore, do not include carriers or by-products that may be included in commercially available materials, unless otherwise specified. The term “weight percent” may be denoted as “wt. %” herein. All ratios are weight ratios unless specifically stated otherwise.

The components and/or steps, including those which may optionally be added, of the various embodiments of the present invention, are described in detail below.

All temperatures are in degrees Celsius, unless specifically stated otherwise.

Except as otherwise noted, all amounts including quantities, percentages, portions, and proportions, are understood to be modified by the word “about,” and amounts are not intended to indicate significant digits.

Except as otherwise noted, the articles “a,” “an,” and “the” mean “one or more.”

Herein, “comprising” means that other steps and other ingredients which do not affect the end result can be added. This term encompasses the terms “consisting of” and “consisting essentially of.” The compositions and methods/processes of the present invention can comprise, consist of, and consist essentially of the essential elements and limitations of the invention described herein, as well as any of the additional or optional ingredients, components, steps, or limitations described herein.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

Herein, “mixtures” is meant to include a simple combination of materials and any compounds that may result from their combination.

By the term “visually distinct,” as used herein, is meant that the regions occupied by each phase can be separately seen by the human eye as distinctly separate regions in contact with one another (i.e., they are not emulsions or dispersions of particles of less than about 100 microns).

By the term “visibly clear,” as used herein, is meant that the transmission of the composition is greater than 60%, preferably greater than 80%. The transparency of the composition is measured using Ultra-Violet/Visible (UV/VIS) Spectrophotometry, which determines the absorption or transmission of UV/IS light by a sample. A light wavelength of 600 nm has been shown to be adequate for characterizing the degree of clarity of cosmetic compositions. Typically, it is best to follow the specific instructions relating the specific spectrophotometer being used. In general, the procedure for measuring percent transmittance starts by setting the spectrophotometer to the 600 nm. Then a calibration “blank” is run to calibrate the readout to 100 percent transmittance. The test sample is then placed in a cuvette designed to fit the specific spectrophotometer and the percent transmittance is measured by the spectrophotometer at 600 nm.

By the term “multi-phased,” or “multi-phase,” as used herein, is meant that at least two phases occupy separate and distinct physical spaces inside the package in which they are stored, but are in direct contact with one another (i.e., they are not separated by a barrier and they are not emulsified). In one preferred embodiment of the present invention, the “multi-phased” personal care compositions comprising at least two phases are present within the container as a visually distinct pattern. The pattern results from the mixing or homogenization of the “multi-phased” composition. The patterns include but are not limited to the following examples: striped, marbled, rectilinear, interrupted striped, checked, mottled, veined, clustered, speckled, geometric, spotted, ribbons, helical, swirled, arrayed, variegated, textured, grooved, ridged, waved, sinusoidal, spiraled, twisted, curved, cycle, streaked, striated, contoured, anisotropic, laced, weave or woven, basket weave, spotted, and tessellated. Preferably the pattern is selected from the group consisting of striped, geometric, marbled, and combinations thereof. In a preferred embodiment the striped pattern may be relatively uniform and even across the dimension of the package. Alternatively, the striped pattern may be uneven, i.e., wavy, or may be non-uniform in dimension. The striped pattern does not need to necessarily extend across the entire dimension of the package. The phases may be various different colors, or include particles, glitter or pearlescence.

The term “personal care composition” as used herein, unless otherwise specified, refers to the compositions of the present invention, wherein the compositions are intended to include only those compositions for topical application to the hair or skin, and specifically excludes those compositions that are directed primarily to other applications such as hard surface cleansing, fabric or laundry cleansing, and similar other applications not intended primarily for topical application to the hair or skin.

The present invention is related to a personal care composition comprising an effective amount of a zinc containing material; a surfactant; a cationic, or a nonionic thickening polymer, or a mixture thereof.

The personal care compositions of the present invention may include, but are not limited to, shampoo, conditioner, antiperspirant, deodorant, hair styling products, cleansers, soaps, bar soap, bodywash, cosmetics, foundations, lotions, creams, ointments, and hydro-alcoholic solutions.

The present invention may be a multi-phase personal care composition comprising: at least two phases wherein at least one of the phases comprises an effective amount of a zinc containing material, at least one surfactant, and at least one cationic or nonionic thickening polymer, or a mixture thereof, and the at least two phases are visually distinct phases that are packaged in physical contact and maintain stability.

In the present invention, the at least one cationic or nonionic thickening polymers, or mixtures thereof, does not bind completely to the zinc ions from the zinc containing material.

In embodiments of the present invention, the pH may be in a range of from about 6.5 to about 12, preferably from about 6.7 to about 9, more preferably from about 6.8 to about 8.2, even more preferably from about 7.0 to about 8.0. In preferred embodiments, the pH of the present invention may be greater than about 6.5, more preferably greater than about 6.8, and still more preferably, greater than about 7.

A. Zinc Containing Material

The composition of the present invention includes an effective amount of a zinc containing material. Herein “zinc containing material” or ZCM means a material comprising zinc bound covalently, and/or ionically, or physically by a host material.

Preferred embodiments of the present invention include an effective amount of a zinc containing material having an aqueous solubility within the composition of less than about 25%, by weight, at 25° C., more preferably less than about 20%; more preferably less than about 15%.

Preferred embodiments of the present invention include from 0.001% to 10% of a zinc containing material; more preferably from 0.01% to 5%; more preferably still from 0.1% to 3%.

In a preferred embodiment, the zinc containing material has an average particle size of from 100 nm to 30 μm.

Examples of zinc containing materials useful in certain embodiments of the present invention include the following:

Inorganic Materials:

Zinc aluminate, Zinc carbonate, Zinc oxide and materials containing zinc oxide (i.e., calamine), Zinc phosphates (i.e., orthophosphate and pyrophosphate), Zinc selenide, Zinc sulfide, Zinc silicates (i.e., ortho- and meta-zinc silicates), Zinc silicofluoride, Zinc borate, Zinc hydroxide and hydroxy sulfate, zinc-containing layered materials and combinations thereof.

Further, layered structures are those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLM's) may have zinc incorporated in the layers and/or as more labile components of the gallery ions.

Many ZLM's occur naturally as minerals. Common examples include hydrozincite (zinc carbonate hydroxide), basic zinc carbonate, aurichalcite (zinc copper carbonate hydroxide), rosasite (copper zinc carbonate hydroxide) and many related minerals that are zinc-containing. Natural ZLM's can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLM's, which are often, but not always, synthetic, is layered doubly hydroxides, which are generally represented by the formula [M²⁺₁₋ₓM³⁺ₓ(OH)₂]^{x+}A^{m-}_{x/m}·nH₂O and some or all of the divalent ions (M²⁺) would be represented as zinc ions (Crepaldi, EL, Pava, P C, Tronto, J, Valim, J B J. Colloid Interfac. Sci. 2002, 248,429-42).

Yet another class of ZLM's can be prepared called hydroxy double salts (Morioka, H., Tagaya, H., Karasu, M, Kadokawa, J, Chiba, K Inorg Chem. 1999, 38, 4211-6). Hydroxy double salts can be represented by the general formula [M²⁺₁₋ₓM²⁺₁₊ₓ(OH)_{3(1-y)}]⁺Aⁿ⁻_{(1=3y)/n}·nH₂O where the two metal ion may be different; if they are the same and represented by zinc, the formula simplifies to [Zn₁₊ₓ(OH)₂]^{2x+}2x A⁻·nH₂O. This latter formula represents (where x=0.4) common materials such as zinc hydroxychloride and zinc hydroxynitrate. These are related to hydrozincite as well wherein the divalent anion is replaced by a monovalent anion. These materials can also be formed in situ in a composition or in or during a production process.

These classes of ZLM's represent relatively common examples of the general category and are not intended to be limiting as to the broader scope of materials which fit this definition.

Natural Zinc Containing Materials/Ores and Minerals:

Sphalerite (zinc blende), Wurtzite, Smithsonite, Franklinite, Zincite, Willemite, Troostite, Hemimorphite and combinations thereof.

Organic Salts:

Zinc fatty acid salts (i.e., caproate, laurate, oleate, stearate, etc.), Zinc salts of alkyl sulfonic acids, Zinc naphthenate, Zinc tartrate, Zinc tannate, Zinc phytate, Zinc monoglycerolate, Zinc allantoinate, Zinc urate, Zinc amino acid salts (i.e., methionate, phenylalinate, tryptophanate, cysteinate, etc.) and combinations thereof.

Polymeric Salts:

Zinc polycarboxylates (i.e., polyacrylate), Zinc polysulfate and combinations thereof.

Physically Adsorbed Forms:

Zinc-loaded ion exchange resins, Zinc adsorbed on particle surfaces, Composite particles in which zinc salts are incorporated, (i.e., as core/shell or aggregate morphologies) and combinations thereof.

Zinc Salts:

zinc oxalate, zinc tannate, zinc tartrate, zinc citrate, zinc oxide, zinc carbonate, zinc hydroxide, zinc oleate, zinc phosphate, zinc silicate, zinc stearate, zinc sulfide, zinc undecylate, and the like, and mixtures thereof; preferably zinc oxide or zinc carbonate basic.

Commercially available sources of zinc oxide include Z-Cote and Z-Cote HPI (BASF), and USP I and USP II (Zinc Corporation of America).

Commercially available sources of zinc carbonate include Zinc Carbonate Basic (Cater Chemicals: Bensenville, IL, USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA).

Zinc Salts That Become Insoluble Above a pH of 7:

zinc acetate, zinc chloride, zinc bromide, zinc fluoride, zinc iodide, zinc sulfate, zinc citrate, zinc lactate, zinc nitrate, zinc propionate, zinc salicylate, zinc tartrate, zinc valerate, zinc gluconate, zinc selenate, zinc benzoate, zinc borate, zinc bromate, zinc formate, zinc glycerophosphate, zinc picrate, zinc butyrate, and the like, and combinations thereof.

Definition of ZCM Solubility:

A zinc containing material with a solubility of less than 25% will have a measurable % soluble zinc value below a threshold value determined by the weight percent and molecular weight of the zinc compound. The theoretical threshold value can be calculated by the following equation (see examples in the Table):

$\frac{\begin{matrix}0.25 * wt . \% Zn Compound in Composition * \\ moles of Zinc in Compound * 65.39 ( MW of Zn )\end{matrix}}{MW of Zn Compound}$

| | | | % soluble Zn+²; |
|---|---|---|---|
| | | | (if 25 wt. % of |
| Zind | | % Zn Compound | zinc source is |
| Compound | Formula | in Composition | soluble)* |
| Zinc Oxide | ZnO | 1.0% | 0.20% |
| Zinc Carbonate | Zn₅(CO₃)₂(OH)₆ | 1.0% | 0.15% |
| Basic | | | |
| (Hydrozincite) | | | |
| Zinc Stearate | Zn(C₁₈H₃₅O₂)₂ | 1.0% | 0.026% |

B. Detersive Surfactant

The composition of the present invention includes a detersive surfactant. The detersive surfactant component is included to provide cleaning performance to the composition. The detersive surfactant component in turn comprises anionic detersive surfactant, zwitterionic or amphoteric detersive surfactant, or a combination thereof. Such surfactants should be physically and chemically compatible with the essential components described herein, or should not otherwise unduly impair product stability, aesthetics or performance.

Suitable anionic detersive surfactant components for use in the composition herein include those which are known for use in hair care or other personal care cleansing compositions. The concentration of the anionic surfactant component in the composition should be sufficient to provide the desired cleaning and lather performance, and generally range from about 5% to about 50%, preferably from about 8% to about 30%, more preferably from about 10% to about 25%, even more preferably from about 12% to about 22%.

Preferred anionic surfactants suitable for use in the compositions are the alkyl and alkyl ether sulfates. These materials have the respective formulae ROSO₃M and RO(C₂H₄O)ₓSO₃M, wherein R is alkyl or alkenyl of from about 8 to about 18 carbon atoms, x is an integer having a value of from 1 to 10, and M is a cation such as ammonium, alkanolamines, such as triethanolamine, monovalent metals, such as sodium and potassium, and polyvalent metal cations, such as magnesium, and calcium.

Preferably, R has from about 8 to about 18 carbon atoms, more preferably from about 10 to about 16 carbon atoms, even more preferably from about 12 to about 14 carbon atoms, in both the alkyl and alkyl ether sulfates. The alkyl ether sulfates are typically made as condensation products of ethylene oxide and monohydric alcohols having from about 8 to about 24 carbon atoms. The alcohols can be synthetic or they can be derived from fats, e.g., coconut oil, palm kernel oil, tallow. Lauryl alcohol and straight chain alcohols derived from coconut oil or palm kernel oil are preferred. Such alcohols are reacted with between about 0 and about 10, preferably from about 2 to about 5, more preferably about 3, molar proportions of ethylene oxide, and the resulting mixture of molecular species having, for example, an average of 3 moles of ethylene oxide per mole of alcohol, is sulfated and neutralized.

Other suitable anionic detersive surfactants are the water-soluble salts of organic, sulfuric acid reaction products conforming to the formula [R¹-SO₃-M] where R¹ is a straight or branched chain, saturated, aliphatic hydrocarbon radical having from about 8 to about 24, preferably about 10 to about 18, carbon atoms; and M is a cation described hereinbefore.

Still other suitable anionic detersive surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil. Other similar anionic surfactants are described in U.S. Pat. Nos. 2,486,921; 2,486,922; and 2,396,278.

Other anionic detersive surfactants suitable for use in the compositions are the succinnates, examples of which include disodium N-octadecylsulfosuccinnate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic detersive surfactants include olefin sulfonates having about 10 to about 24 carbon atoms. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A non limiting example of such an alpha-olefin sulfonate mixture is described in U.S. Pat. No. 3,332,880.

Another class of anionic detersive surfactants suitable for use in the compositions are the beta-alkyloxy alkane sulfonates. These surfactants conform to the formula 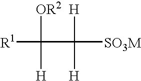
where R¹ is a straight chain alkyl group having from about 6 to about 20 carbon atoms, R² is a lower alkyl group having from about 1 to about 3 carbon atoms, preferably 1 carbon atom, and M is a water-soluble cation as described hereinbefore.

Preferred anionic detersive surfactants for use in the compositions include sodium lauryl sulfate, sodium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, ammonium lauryl sulfate, ammonium laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, monoethanolamine lauryl sulfate, sodium tridecyl benzene sulfonate, sodium dodecyl benzene sulfonate, sodium cocoyl isethionate, and combinations thereof. Most preferred anionic detersive surfactants include sodium lauryl sulfate and sodium laureth sulfate.

Suitable amphoteric or zwitterionic detersive surfactants for use in the composition herein include those which are known for use in hair care or other personal care cleansing. Concentration of such amphoteric detersive surfactants preferably ranges from about 0.5% to about 20%, preferably from about 1% to about 10%. Non limiting examples of suitable zwitterionic or amphoteric surfactants are described in U.S. Pat. Nos. 5,104,646 (Bolich Jr. et al.) and 5,106,609 (Bolich Jr. et al.).

Amphoteric detersive surfactants suitable for use in the composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Preferred amphoteric detersive surfactants for use in the present invention include cocoamphoacetate, cocoamphodiacetate, lauroamphoacetate, lauroamphodiacetate, and mixtures thereof.

Zwitterionic detersive surfactants suitable for use in the composition are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate, or phosphonate. Zwitterionics such as betaines are preferred.

The compositions of the present invention may further comprise additional surfactants for use in combination with the anionic detersive surfactant component described hereinbefore. Suitable optional surfactants include nonionic and cationic surfactants. Any such surfactant known in the art for use in hair or personal care products may be used, provided that the optional additional surfactant is also chemically and physically compatible with the essential components of the composition, or does not otherwise unduly impair product performance, aesthetics or stability. The concentration of the optional additional surfactants in the composition may vary with the cleansing or lather performance desired, the optional surfactant selected, the desired product concentration, the presence of other components in the composition, and other factors well known in the art.

Non limiting examples of other anionic, zwitterionic, amphoteric, or optional additional surfactants suitable for use in the compositions are described in McCutcheon's, Emulsifiers and Detergents, 2002 Annual, published by M. C. Publishing Co., and U.S. Pat. Nos. 3,929,678; 2,658,072; 2,438,091; and 2,528,378.

C. Thickening Polymer

The cleansing phase of the present invention comprises at least one thickener. Preferred thickeners are selected from the group consisting of inorganic water thickeners, polymeric thickeners, additives that promote thickening via lamellar structuring of surfactants, organic crystalline thickeners, and mixtures thereof.

The thickener in the present invention can be hydrophilic. The amount of thickener present may be less than about 20%, preferably less than about 10%, and even more preferably less than about 5%.

Non-limiting examples of inorganic water thickeners for use in the personal care composition include silicas, clays such as a synthetic silicates (Laponite XLG and Laponite XLS from Southern Clay), or mixtures thereof.

Additional non-limiting examples of polymeric thickeners for use in the personal care composition include cellulosic gel, hydroxypropyl starch phosphate (Structure XL from National Starch), polyvinyl alcohol, or mixtures thereof. Further, non-limiting examples of polymeric thickeners for use in the personal care composition include synthetic and natural gums and thickeners such as xanthan gum (Ketrol CG-T from CP Kelco), succinoglycan (Rheozan from Rhodia), gellum gum, pectin, alginates, starches including pregelatinized starches, modified starches, or mixtures thereof, acrylates/aminoacrylates/CD-30 alkyl PEG-20 itaconate copolymer (Structure Plus from National Starch).

Non-limiting examples of additives that promote thickening via lamellar structuring of surfactants for use in the personal care composition include fatty amides, fatty alcohols, fatty acid or ester derivatives thereof, electrolytes, and mixtures thereof. Examples of fatty acids which may be used are C₁₀-C₂₂ acids such as the following: lauric acid, oleic acid, isostearic acid, linoleic acid, linolenic acid, ricinoleic acid, elaidic acid, arichidonic acid, myristoleic acid, palmitoleic acid, and the like. Ester derivatives include propylene glycol, isostearate, propylene glycol oleate, glyceryl isostearate, glyceryl oleate, polyglyceryl diisostearate, and the like.

Non-limiting examples of organic crystalline thickeners for use in the personal care composition include ethylene glycol esters of fatty acids preferably having from about 16 to about 22 carbon atoms. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin R available from Rheox, Inc. Other suitable thickeners are alkyl (C16 to C22) dimethyl amide oxides such as stearyl dimethyl amine oxide. Also useful herein are long chain acyl derivatives, ethylene glycol esters of long chain carboxylic acids, long chain amine oxides, and alkanol amides of long chain carboxylic acids.

In the present invention, the cationic or nonionic thickening polymers, or mixtures thereof, does not bind completely to the zinc ions from the zinc containing material.

1. Cationic Thickening Polymers

The compositions of the present invention may contain a cationic polymer. Preferred cationic polymers will have cationic charge densities of at least about 0.9 meq/gm, preferably at least about 1.2 meq/gm, more preferably at least about 1.5 meq/gm, but also preferably less than about 7 meq/gm, more preferably less than about 5 meq/gm, at the pH of intended use of the composition, which pH will generally range from about pH 3 to about pH 9, preferably between about pH 4 and about pH 8. Herein, “cationic charge density” of a polymer refers to the ratio of the number of positive charges on the polymer to the molecular weight of the polymer. The average molecular weight of such suitable cationic polymers will generally be between about 10,000 and 10 million, preferably between about 50,000 and about 5 million, more preferably between about 100,000 and about 3 million.

Suitable cationic polymers for use in the compositions of the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the composition. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair product performance, stability, or aesthetics. Non limiting examples of such counterions include halides (e.g., chloride, fluoride, bromide, iodide), sulfate, and methylsulfate.

Non limiting examples of such polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)).

Non limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, or vinyl pyrrolidone.

Suitable cationic protonated amino and quaternary ammonium monomers, for inclusion in the cationic polymers of the composition herein, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Other suitable cationic polymers for use in the compositions include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-11); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquatemium 6 and Polyquaternium 7, respectively); amphoteric copolymers of acrylic acid including copolymers of acrylic acid and dimethyldiallylammonium chloride (referred to in the industry by CTFA as Polyquatemium 22), terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methylacrylate (referred to in the industry by CTFA as Polyquatemium 47). Preferred cationic substituted monomers are the cationic substituted dialkylaminoalkyl acrylamides, dialkylaminoalkyl methacrylamides, and combinations thereof. These preferred monomers conform the to the formula 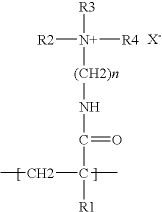
wherein R¹ is hydrogen, methyl or ethyl; each of R², R³, and R⁴ are independently hydrogen or a short chain alkyl having from about 1 to about 8 carbon atoms, preferably from about 1 to about 5 carbon atoms, more preferably from about 1 to about 2 carbon atoms; n is an integer having a value of from about 1 to about 8, preferably from about 1 to about 4; and X is a counterion. The nitrogen attached to R², R³ and R⁴ may be a protonated amine (primary, secondary, or tertiary), but is preferably a quaternary ammonium wherein each of R², R³, and R⁴ are alkyl groups a non limiting example of which is polymethyacrylamidopropyl trimonium chloride, available under the trade name Polycare 133, from Rhone-Poulenc, Cranberry, N.J., U.S.A.

Other suitable cationic polymers for use in the composition include polysaccharide polymers, such as cationic cellulose derivatives and cationic starch derivatives. Suitable cationic polysaccharide polymers include those which conform to the formula 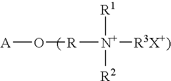
wherein A is an anhydroglucose residual group, such as a starch or cellulose anhydroglucose residual; R is an alkylene oxyalkylene, polyoxyalkylene, or hydroxyalkylene group, or combination thereof; R¹, R², and R³ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R², and R³) preferably being about 20 or less; and X is an anionic counterion as described in hereinbefore.

Preferred cationic cellulose polymers are salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers. Other suitable types of cationic cellulose includes the polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename Polymer LM-200.

Other suitable cationic polymers include cationic guar gum derivatives, such as guar hydroxypropyltrimonium chloride, specific examples of which include the Jaguar series commercially available from Rhone-Poulenc Incorporated and the N-Hance series commercially available from Aqualon Division of Hercules, Inc. Other suitable cationic polymers include quaternary nitrogen-containing cellulose ethers, some examples of which are described in U.S. Pat. No. 3,962,418. Other suitable cationic polymers include copolymers of etherified cellulose, guar and starch, some examples of which are described in U.S. Pat. No. 3,958,581. When used, the cationic polymers herein are either soluble in the composition or are soluble in a complex coacervate phase in the composition formed by the cationic polymer and the anionic, amphoteric and/or zwitterionic detersive surfactant component described hereinbefore. Complex coacervates of the cationic polymer can also be formed with other charged materials in the composition.

Techniques for analysis of formation of complex coacervates are known in the art. For example, microscopic analyses of the compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition.

2. Nonionic Thickening Polymers

The compositions of the present invention may contain a nonionic polymer. Non-limiting examples of nonionic polymers for use in the personal care composition include methyl hydroxypropyl cellulose, xanthan gum, alhinate polysaccharide Gellan Gum (Kelcogel from CP Kelco), polysaccharide gum, hydroxyl propyl cellulose (Methocel from Dow/Amerchol), hydroxyl propyl methyl cellulose (Klucel from Hercules), hydroxyl ethyl cellulose, polyalkylene gylcols, and mixtures thereof. Particularly useful nonionic polymers include polysaccharide gum, hydroxyl propyl cellulose, hydroxyl propyl methyl cellulose, or combinations thereof.

Polyalkylene glycols having a molecular weight of more than about 1000 are useful herein. Useful are those having the following general formula: 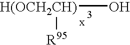
wherein R⁹⁵ is selected from the group consisting of H, methyl, and mixtures thereof. Polyethylene glycol polymers useful herein are PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

3. Rheology of Thickeners

The thickening polymers that are useful in the present invention are those which can provide appropriate viscosity and rheology properties to the composition, so that the composition of the present composition has a suitable viscosity from at least about 15 Pas to about 20000 Pas at a shear rate of about 0.0005 s⁻¹ to about 0.005 s^{-1.} Preferably, the composition has a viscosity from at least about 25 Pas to about 10000 Pas, more preferably from at least about 40 Pas to about 5000 Pas, even more preferably from at least about 50 Pas to about 2000 Pas at a shear rate of about 0.0005 s⁻¹ to about 0.005 s-l. At a high shear rate of about 10 s⁻¹ to about 1000 s⁻¹, the viscosity is about 0.001 Pas to about 50 Pas, preferably, from about 0.01 Pas to about 20 Pas, more preferably, the viscosity is about 1 Pas to about 15 Pas

D. Carrier

The compositions of the present invention may also comprise a carrier. The compositions of the present invention are typically in the form of pourable liquids (under ambient conditions).

The compositions will therefore typically comprise an aqueous carrier, which is present at a level of from about 20% to about 95%, preferably from about 60% to about 85%. The aqueous carrier may comprise water, or a miscible mixture of water and organic solvent, but preferably comprises water with minimal or no significant concentrations of organic solvent, except as otherwise incidentally incorporated into the composition as minor ingredients of other essential or optional components.

In a preferred embodiment, the carrier is water. Preferably the compositions of the present invention comprise from 40% to 95% water by weight of the composition; preferably from 50% to 85%, more preferably still from 60% to 80%.

The composition of the present invention may be in the form of topical compositions, which includes a topical carrier. Preferably, the topical carrier is selected from a broad range of traditional personal care carriers depending on the type of composition to be formed. By suitable selections of compatible carriers, it is contemplated that such a composition is prepared in the form of daily skin or hair products including conditioning treatments, cleansing products, such as hair and/or scalp shampoos, body washes, hand cleansers, water-less hand sanitizer/cleansers, facial cleansers, and the like.

E. Additional Components

1. Zinc lonophoric Material (ZIM)

In another embodiment of the present invention, the composition further includes a zinc ionophoric material. Herein, “zinc ionophoric material” and “ZIM” means a material which is or forms a hydrophobic molecule capable of increasing cell permeability to zinc ions (i.e., exhibiting zinc ionophoric behavior). Without being bound by theory, it is believed that ZIMs shield the charge of the zinc ion to be transported, enabling it to penetrate the hydrophobic interior of the lipid bilayer. ZIMs may be channel-forming ionophores or mobile ion carriers.

ZIMs may be those commonly known as zinc ionophores or those that are hydrophobic zinc chelators that possess zinc ionophoric behavior. Hydrophobic zinc chelators are materials that bind zinc and increase hydrophobicity of zinc ions such that, for example, it will partition into non-aqueous solvents. ZIMs can be effective including zinc being present in the composition or zinc being available within the system wherein a ZIM is present, yet preferred ZIMs contain zinc ions; i.e., zinc salt forms of materials exhibiting zinc ionophoric behavior.

Preferred embodiments include from 0.01% to 5% of a ZIM; more preferably from 0.1% to 2%.

In embodiments having a zinc containing material and a ZIM, the ratio of zinc containing material to ZIM is preferably from 5:100 to 5:1; more preferably from about 2:10 to 3:1; more preferably still from 1:2 to 2:1.

In preferred embodiments of the present invention, the ZIM has a potency against target microorganisms such that the minimum inhibitory concentration (“MIC”) is below 5000 parts per million. The MIC is a measurement well understood by those skilled in the art and is indicative of anti-fungal efficacy. Generally, the lower the value of the composition, the better its anti-fungal efficacy, due to increased inherent ability of the anti-dandruff agent to inhibit the growth of microorganisms. The lowest tested dilution of anti-microbial active that yields no growth is defined as the MIC.

Examples of ZIMs useful in embodiments of the present invention include the following:

| Class | Name (Synonyms) | Structure |
|---|---|---|
| Bio-molecules, Peptides and Naturally Occurring Materials and derivatives thereof having zinc ionophoric behavior | Lasalocid (X537A) A23187 (Calcimycin) 4-Br A23187 Ionomycin Cyclosporin | |
| #A | 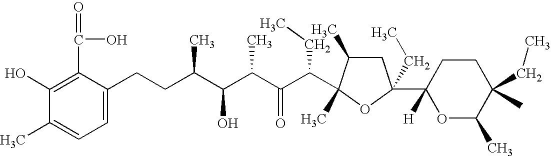 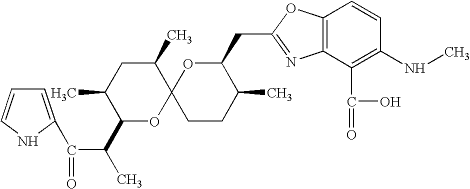 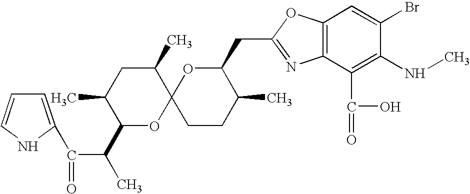 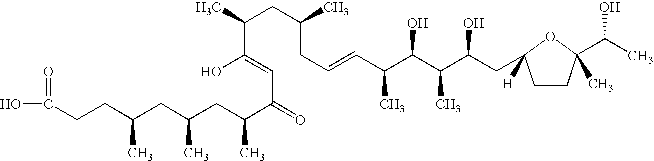 | |
| | Cyclosporin A | Cyclic undecapeptide: |
| | | cyclo-(MeBMT-Abu-Sar-MeLeu-Val-MeLeu-Ala-D-Ala-MeLeu-MeLeu-MeVal) |
| Hydroxy- quinolines | Diodoquin (Iodoquinol; 5,7-Di- iodo-8-hy- droxyquinoline) | 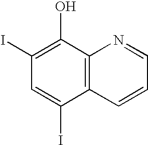 |
| | Enterovioform (Iodochloro hydroxyquinoline; 5-Cl, 7-I-8-hydroxy- quinoline) | 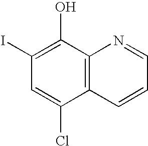 |
| | Sterosan (Chloroquinaldol; 2-Me, 5,7-Dichloro-8-hy- droxyquinoline) | 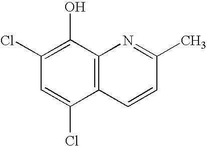 |
| | 5-7-Bibromo-8-hy- droxyquinoline | 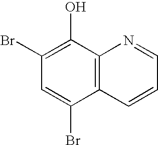 |
| Sulfur-Based Compounds | Tetra-n-butyl thiuram Disulfide (TBTDS) | 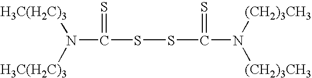 |
| Transport | Albumin, histidine, | |
| Enhancers | arachidonic acid, | |
| | picolinic acid, | |
| | dihydroxyvitamin | |
| | D₃, ethylmaltol | |

In a preferred embodiment, the ZIM is pyrithione or a polyvalent metal salt of pyrithione. Any form of polyvalent metal pyrithione salts may be used, including platelet and needle structures. Preferred salts for use herein include those formed from the polyvalent metals magnesium, barium, bismuth, strontium, copper, zinc, cadmium, zirconium and mixtures thereof, more preferably zinc. Even more preferred for use herein is the zinc salt of 1-hydroxy-2-pyridinethione (known as “zinc pyrithione” or “ZPT”); more preferably ZPT in platelet particle form, wherein the particles have an average size of up to about 201 μm, preferably up to about 5 μm, more preferably up to about 2.5 μm.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

It is further contemplated that when ZPT is used as the anti-microbial particulate in the anti-microbial compositions herein, that an additional benefit of hair growth or re-growth may be stimulated or regulated, or both, or that hair loss may be reduced or inhibited, or that hair may appear thicker or fuller.

Zinc pyrithione may be made by reacting 1-hydroxy-2-pyridinethione (i.e., pyrithione acid) or a soluble salt thereof with a zinc salt (e.g., zinc sulfate) to form a zinc pyrithione precipitate, as illustrated in U.S. Pat. No. 2,809,971.

2. Anti-Fungal or Anti-Microbial Actives

The compositions of the present invention may further include one or more anti-fungal or anti-microbial actives. Suitable anti-microbial actives include coal tar, sulfur, whitfield's ointment, castellani's paint, aluminum chloride, gentian violet, octopirox (piroctone olamine), ciclopirox olamine, undecylenic acid and it's metal salts, potassium permanganate, selenium sulfide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Preferred anti-microbials include itraconazole, ketoconazole, selenium sulfide and coal tar.

a. Azoles

Azole anti-microbials include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in the composition, the azole anti-microbial active is included in an amount from about 0.01% to about 5%, preferably from about 0.1% to about 3%, and more preferably from about 0.3% to about 2%, by weight of the composition. Especially preferred herein is ketoconazole.

b. Selenium Sulfide

Selenium sulfide is a particulate anti-dandruff agent suitable for use in the anti-microbial compositions of the present invention, effective concentrations of which range from about 0.1% to about 4%, by weight of the composition, preferably from about 0.3% to about 2.5%, more preferably from about 0.5% to about 1.5%. Selenium sulfide is generally regarded as a compound having one mole of selenium and two moles of sulfur, although it may also be a cyclic structure that conforms to the general formula SeₓS_{y}, wherein x+y =8. Average particle diameters for the selenium sulfide are typically less than 15 μm, as measured by forward laser light scattering device (e.g., Malvern 3600 instrument), preferably less than 10 μm. Selenium sulfide compounds are described, for example, in U.S. Pat. No. 2,694,668; U.S. Pat. No. 3,152,046; U.S. Pat. No. 4,089,945; and U.S. Pat. No. 4,885,107.

c. Sulfur

Sulfur may also be used as a particulate anti-microbial/anti-dandruff agent in the anti-microbial compositions of the present invention. Effective concentrations of the particulate sulfur are typically from about 1% to about 4%, by weight of the composition, preferably from about 2% to about 4%.

d. Keratolytic Agents

The present invention may further comprise one or more keratolytic agents such as Salicylic Acid.

Additional anti-microbial actives of the present invention may include extracts of melaleuca (tea tree) and charcoal. The present invention may also comprise combinations of anti-microbial actives. Such combinations may include octopirox and zinc pyrithione combinations, pine tar and sulfur combinations, salicylic acid and zinc pyrithione combinations, octopirox and climbasole combinations, and salicylic acid and octopirox combinations, and mixtures thereof.

3. Optional Ingredients

The present invention may, in some embodiments, further comprise additional optional components known or otherwise effective for use in hair care or personal care products. The concentration of such optional ingredients generally ranges from zero to about 25%, more typically from about 0.05% to about 20%, even more typically from about 0.1% to about 15%, by weight of the composition. Such optional components should also be physically and chemically compatible with the essential components described herein, and should not otherwise unduly impair product stability, aesthetics, or performance.

Non-limiting examples of optional components for use in the present invention include anti-static agents (water-insoluble cationic surfactants), dispersed particles, foam boosters (fatty ester (e.g., C₈-C₂₂) mono- and di (C₁-C₅, especially C₁-C₃) alkanol amides), anti-dandruff agents in addition to the anti-dandruff agents described above, viscosity adjusting agents and thickeners, suspension materials (e.g., EGDS, thixins), pH adjusting agents (e.g., sodium citrate, citric acid, succinic acid, sodium succinate, sodium maleate, sodium glycolate, malic acid, glycolic acid, hydrochloric acid, sulfuric acid, sodium bicarbonate, sodium hydroxide, and sodium carbonate), chelants, preservatives (e.g., DMDM hydantoin), anti-microbial agents (e.g., triclosan or triclocarbon), dyes, organic solvents or diluents, pearlescent aids, perfumes, fatty alcohols, proteins, skin active agents, sunscreens, UV absorbers, vitamins (such as retinoids including retinyl propionate, vitamin E such as tocopherol acetate, panthenol, and vitamin B3 compounds including niacinamide), minerals, emulsifiers, volatile carriers, select stability actives, styling polymers, organic styling polymers, silicone-grafted styling polymers, cationic spreading agents, pediculocides, polyalkylene glycols, conditioning agents (hydrocarbon oils, fatty esters, silicones), additional surfactants or nonionic cosurfactants, herbal/fruit/food extracts, sphingolipids derivatives or synthetical derivative, clay, and combinations thereof.

The compositions of the present invention may contain other vitamins and amino acids such as: water soluble vitamins such as vitamin B 1, B2, B6, B 12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

The compositions of the present invention may also contain pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names.

### Methods of Making

The compositions of the present invention may be prepared by any known or otherwise effective technique, suitable for providing a personal care composition. Methods for preparing the anti-dandruff and conditioning shampoo embodiments of the present invention include conventional formulation and mixing techniques. A method such as that described in U.S. Pat. No. 5,837,661, could be employed, wherein the anti-microbial agent of the present invention would typically be added in the same step as the silicone premix is added in the U.S. Pat. No. 5,837,661 description.

The composition of the present invention may be prepared by any known or otherwise effective technique, suitable for making and formulating the desired multi-phase product form. It is especially effective to combine toothpaste-tube filling technology with a spinning stage design.

### Methods of Use

The compositions of the present invention may be used in direct application to the skin or in a conventional manner for cleansing skin and hair and controlling microbial infection (including fungal, viral, or bacterial infections) on the skin or scalp. The compositions herein are useful for cleansing the hair and scalp, and other areas of the body such as underarm, feet, and groin areas and for any other area of skin in need of treatment. The present invention may be used for treating or cleansing of the skin or hair of animals as well. The present invention may also have healing and repairing properties for treating damaged skin. An effective amount of the composition, typically from about 1 g to about 50 g, preferably from about 1 g to about 20 g of the composition, for cleansing hair, skin or other area of the body, is topically applied to the hair, skin or other area that has preferably been wetted, generally with water, and then rinsed off. Application to the hair typically includes working the shampoo composition through the hair.

A preferred method for providing anti-microbial (especially anti-dandruff) efficacy with a shampoo embodiment comprises the steps of: (a) wetting the hair with water, (b) applying an effective amount of the anti-microbial shampoo composition to the hair, and (c) rinsing the anti-microbial shampoo composition from the hair using water. These steps may be repeated as many times as desired to achieve the cleansing, conditioning, and anti-microbial/anti-dandruff benefits sought.

It is also contemplated that when the anti-microbial active employed is zinc pyrithione, and/or if other optional hair growth regulating agents are employed, the anti-microbial compositions of the present invention, may, provide for the regulation of growth of the hair. The method of regularly using such shampoo compositions comprises repeating steps a, b, and c (above).

A further embodiment of the present invention comprises a method of treating athlete's foot comprising the use of the composition according to the present invention, a method of treating microbial infections comprising the use of composition as described herein, method of improving the appearance of a scalp comprising the use of the composition according present invention, a method of treating fungal infections comprising the use of the composition according to the present invention, a method of treating dandruff comprising the use of the composition of the present invention, a method of treating diaper dermatitis and candidiasis comprising the use of the compositions of the present invention as described herein, a method of treating tinea capitis comprising the use of the composition according to the present invention, a method of treating yeast infections comprising the use of the composition according to the present invention, a method of treating onychomycosis comprising the use of the composition according to the present invention.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its scope. Ingredients are identified by chemical or CTFA name, or otherwise defined below.

The composition of the invention can be made by mixing one or more selected metal ion sources and one or more metal salts of pyrithione in an appropriate media or carrier, or by adding the individual components separately to the skin or hair cleansing compositions. Useful carriers are discussed more fully above.

### Examples 1-10

| Thickened AD Formulas | | | | | | | | | |
|---|---|---|---|---|---|---|---|---|---|
| 1 | 2 | 3 | 4 | 5 | 6 | 7 | 8 | 9 | 10 |

Phase A Composition

| | | | | | | | | | | |
|---|---|---|---|---|---|---|---|---|---|---|
| Surfactants | | | | | | | | | | |
| Sodium Laureth | 10.00 | 10.00 | 10.00 | 10.00 | 10.00 | 10.00 | 10.00 | 10.00 | 10.00 | 10.00 |
| Sulfate (SLE3S) | | | | | | | | | | |
| Sodium Lauryl | 6.00 | 6.00 | 6.00 | 6.00 | 6.00 | 6.00 | 6.00 | 6.00 | 6.00 | 6.00 |
| Sulfate (SLS) | | | | | | | | | | |
| Polymers | | | | | | | | | | |
| Guar Hydroxy Propyl | | | | | 0.500 | 0.500 | 0.500 | 0.500 | 0.125 | |
| Trimonium Chloride¹ | | | | | | | | | | |
| Polyquaterium-10² | 0.400 | 0.500 | 0.500 | 0.500 | | | | | | 0.500 |
| PEG-7M | | | | | | | | | | 0.100 |
| Gellan Gum³ | 2.000 | 1.000 | | | 2.000 | 1.000 | | | 2.000 | 2.000 |
| Hydroxypropyl | | | 2.000 | | | | 2.000 | | | |
| Cellulose⁴ | | | | | | | | | | |
| Hydroxypropyl | | 1.000 | | 2.000 | | 1.000 | | 2.000 | | |
| Methylcellulose⁵ | | | | | | | | | | |
| Silicones | | | | | | | | | | |
| Dimethicone⁶ | 0.85 | 0.00 | 1.40 | 1.40 | 1.00 | 0.85 | 0.85 | 0.00 | 0.40 | 0.55 |
| AD Active | | | | | | | | | | |
| ZPT (U2 ZPT) | 1.000 | 0.000 | 1.000 | 1.000 | 1.000 | 1.000 | 1.000 | 1.000 | 1.000 | 1.000 |
| Suspending/Pearlizing | | | | | | | | | | |
| Agent | | | | | | | | | | |
| Glycol Distearate | 1.50 | 0.00 | 1.50 | 1.50 | 1.50 | 1.50 | 1.50 | 1.50 | 1.50 | 1.50 |
| Others | | | | | | | | | | |
| Basic Zinc Carbonate⁷ | 1.610 | 0.000 | 1.610 | 1.610 | 1.610 | 1.610 | 1.610 | 0.000 | 1.610 | 1.610 |
| CMEA | 1.600 | 1.600 | 1.600 | 1.600 | 1.600 | 1.600 | 1.600 | 1.600 | 1.600 | 1.600 |
| Cetyl Alcohol | 0.600 | 0.600 | 0.600 | 0.600 | 0.600 | 0.600 | 0.600 | 0.600 | 0.600 | 0.600 |
| Hydrochloric | 0.42 | 0.42 | 0.42 | 0.42 | 0.42 | 0.42 | 0.42 | 0.42 | 0.42 | 0.42 |
| Acid* | | | | | | | | | | |
| Magnesium | 0.28 | 0.28 | 0.28 | 0.28 | 0.28 | 0.28 | 0.28 | 0.28 | 0.28 | 0.28 |
| Sulfate | | | | | | | | | | |
| Sodium | 0.800 | 0.800 | 0.800 | 0.800 | 0.800 | 0.800 | 0.800 | 0.800 | 0.800 | 0.800 |
| Chloride** | | | | | | | | | | |
| Sodium | 0.00 | 0.00 | 0.00 | 0.00 | 0.00 | 0.00 | 0.00 | 0.00 | 0.00 | 0.00 |
| Xylenesulfonate** | | | | | | | | | | |
| Perfume | 0.700 | 0.750 | 0.700 | 0.350 | 0.700 | 0.750 | 0.700 | 1.000 | 0.650 | 0.730 |
| Sodium Benzoate | 0.250 | 0.250 | 0.250 | 0.250 | 0.250 | 0.250 | 0.250 | 0.250 | 0.250 | 0.250 |
| Kathon | 7.5E−04 | 7.5E−04 | 7.5E−04 | 7.5E−04 | 7.5E−04 | 7.5E−04 | 7.5E−04 | 7.5E−04 | 7.5E−04 | 7.5E−04 |
| Benzyl Alcohol | 0.0225 | 0.0225 | 0.0225 | 0.0225 | 0.0225 | 0.0225 | 0.0225 | 0.0225 | 0.0225 | 0.0225 |
| Water | Q.S. | Q.S. | Q.S. | Q.S. | Q.S. | Q.S. | Q.S. | Q.S. | Q.S. | Q.S. |

Phase B Composition

| | | | | | | | | | | |
|---|---|---|---|---|---|---|---|---|---|---|
| Sodium Laureth | 10.00 | 10.00 | 10.00 | 10.00 | 10.00 | 10.00 | 10.00 | 10.00 | 10.00 | 10.00 |
| Sulfate (SLE3S) | | | | | | | | | | |
| Sodium Lauryl | 6.00 | 6.00 | 6.00 | 6.00 | 6.00 | 6.00 | 6.00 | 6.00 | 6.00 | 6.00 |
| Sulfate (SLS) | | | | | | | | | | |
| Polymers | | | | | | | | | | |
| Guar Hydroxy Propyl | | | | | 0.500 | 0.500 | 0.500 | 0.500 | 0.125 | |
| Trimonium Chloride¹ | | | | | | | | | | |
| Polyquaterium-10² | 0.400 | 0.500 | 0.500 | 0.500 | | | | | | 0.500 |
| PEG-7M | | | | | | | | | | 0.100 |
| Gellan Gum³ | 2.000 | 1.000 | | | 2.000 | 1.000 | | | 1.000 | 2.000 |
| Hydroxypropyl | | | 2.000 | | | | 2.000 | | | |
| Cellulose⁴ | | | | | | | | | | |
| Hydroxypropyl | | 1.000 | | 2.000 | | 1.000 | | 2.000 | 1.000 | |
| Methylcellulose⁵ | | | | | | | | | | |
| Silicones | | | | | | | | | | |
| Dimethicone⁶ | 0.85 | 2.55 | 1.40 | 1.40 | 1.00 | 0.85 | 0.85 | 1.00 | 0.40 | 0.55 |
| AD Active | | | | | | | | | | |
| ZPT (U2 ZPT) | 1.000 | 3.000 | 1.000 | 1.000 | 1.000 | 1.000 | 1.000 | 1.500 | 1.000 | 1.000 |
| Suspending/Pearlizing | | | | | | | | | | |
| Agent | | | | | | | | | | |
| Glycol Distearate | 1.50 | 1.50 | 1.50 | 1.50 | 1.50 | 1.50 | 1.50 | 1.50 | 1.50 | 1.50 |
| Others | | | | | | | | | | |
| Basic Zinc | 1.610 | 4.830 | 1.610 | 1.610 | 1.610 | 1.610 | 1.610 | 2.415 | 1.610 | 1.610 |
| Carbonate⁷ | | | | | | | | | | |
| CMEA | 1.600 | 1.600 | 1.600 | 1.600 | 1.600 | 1.600 | 1.600 | 1.600 | 1.600 | 1.600 |
| Cetyl Alcohol | 0.600 | 0.600 | 0.600 | 0.600 | 0.600 | 0.600 | 0.600 | 0.600 | 0.600 | 0.600 |
| Hydrochloric | 0.42 | 0.42 | 0.42 | 0.42 | 0.42 | 0.42 | 0.42 | 0.42 | 0.42 | 0.42 |
| Acid* | | | | | | | | | | |
| Magnesium | 0.28 | 0.28 | 0.28 | 0.28 | 0.28 | 0.28 | 0.28 | 0.28 | 0.28 | 0.28 |
| Sulfate | | | | | | | | | | |
| Sodium | 0.800 | 0.800 | 0.800 | 0.800 | 0.800 | 0.800 | 0.800 | 0.800 | 0.800 | 0.800 |
| Chloride** | | | | | | | | | | |
| Sodium Xylenesulfonate** | 0.00 | 0.00 | 0.00 | 0.00 | 0.00 | 0.00 | 0.00 | 0.00 | 0.00 | 0.00 |
| Perfume | 0.700 | 0.750 | 0.700 | 0.350 | 0.700 | 0.750 | 0.700 | 1.000 | 0.650 | 0.730 |
| Sodium Benzoate | 0.250 | 0.250 | 0.250 | 0.250 | 0.250 | 0.250 | 0.250 | 0.250 | 0.250 | 0.250 |
| Kathon | 7.5E−04 | 7.5E−04 | 7.5E−04 | 7.5E−04 | 7.5E−04 | 7.5E−04 | 7.5E−04 | 7.5E−04 | 7.5E−04 | 7.5E−04 |
| Benzyl Alcohol | 0.0225 | 0.0225 | 0.0225 | 0.0225 | 0.0225 | 0.0225 | 0.0225 | 0.022 5 | 0.0225 | 0.0225 |
| Water | Q.S. | Q.S. | Q.S. | Q.S. | Q.S. | Q.S. | Q.S. | Q.S. | Q.S. | Q.S. |
| Ratio of Phase A | 60:40 | 67:33 | 70:30 | 70:30 | 90:10 | 50:50 | 25:75 | 33:67 | 50:50 | 70:30 |
| to Phase B | | | | | | | | | | |

*adjustable to achieve target pH
**adjustable to achieve target viscosity
¹Available as N-Hance 3269 from Aqualon
²Available as LR400 from Dow/Amerchol
³Available as Kelcogel from CP Kelco
⁴Available as Methocel from Dow/Amerchol
⁵Available as Klucel from Hercules
⁶Available as Viscasil 330M from GE Silicones
⁷Available as Bruggemann ZC from Bruggemann Chemicals

| | Thickened AD Formulas | | | | | | | | | |
|---|---|---|---|---|---|---|---|---|---|---|
| | 11 | 12 | 13 | 14 | 15 | 16 | 17 | 18 | 19 | 20 |
| Surfactants | | | | | | | | | | |
| Sodium Laureth | 10.00 | 10.00 | 10.00 | 10.00 | 10.00 | 10.00 | 10.00 | 10.00 | 10.00 | 10.00 |
| Sulfate (SLE3S) | | | | | | | | | | |
| Sodium Lauryl | 6.00 | 6.00 | 6.00 | 6.00 | 6.00 | 6.00 | 6.00 | 6.00 | 6.00 | 6.00 |
| Sulfate (SLS) | | | | | | | | | | |
| Polymers | | | | | | | | | | |
| Guar Hydroxy | | | | | 0.500 | 0.500 | 0.500 | 0.500 | 0.125 | |
| Propyl | | | | | | | | | | |
| Trimonium | | | | | | | | | | |
| Chloride¹ | | | | | | | | | | |
| Polyquaterium-10² | 0.400 | 0.500 | 0.500 | 0.500 | | | | | | 0.500 |
| PEG-7M | | | | | | | | | | 0.100 |
| Gellan Gum³ | 2.000 | 1.000 | | | 2.000 | 1.000 | | | 2.000 | 2.000 |
| Hydroxypropyl | | | 2.000 | | | | 2.000 | | | |
| Cellulose⁴ | | | | | | | | | | |
| Hydroxypropyl | | 1.000 | | 2.000 | | 1.000 | | 2.000 | | |
| Methylcellulose⁵ | | | | | | | | | | |
| Silicones | | | | | | | | | | |
| Dimethicone⁶ | 0.85 | 0.00 | 1.40 | 1.40 | 1.00 | 0.85 | 0.85 | 0.00 | 0.40 | 0.55 |
| AD Active | | | | | | | | | | |
| ZPT (U2 ZPT) | 1.000 | 0.000 | 1.000 | 1.000 | 1.000 | 1.000 | 1.000 | 1.000 | 1.000 | 1.000 |
| Suspending/Pearl- | | | | | | | | | | |
| izing Agent | | | | | | | | | | |
| Glycol Distearate | 1.50 | 0.00 | 1.50 | 1.50 | 1.50 | 1.50 | 1.50 | 1.50 | 1.50 | 1.50 |
| Others | | | | | | | | | | |
| Basic Zinc | 1.610 | 0.000 | 1.610 | 1.610 | 1.610 | 1.610 | 1.610 | 0.000 | 1.610 | 1.610 |
| Carbonate⁷ | | | | | | | | | | |
| CMEA | 1.600 | 1.600 | 1.600 | 1.600 | 1.600 | 1.600 | 1.600 | 1.600 | 1.600 | 1.600 |
| Cetyl Alcohol | 0.600 | 0.600 | 0.600 | 0.600 | 0.600 | 0.600 | 0.600 | 0.600 | 0.600 | 0.600 |
| Hydrochloric | 0.42 | 0.42 | 0.42 | 0.42 | 0.42 | 0.42 | 0.42 | 0.42 | 0.42 | 0.42 |
| Acid* | | | | | | | | | | |
| Magnesium | 0.28 | 0.28 | 0.28 | 0.28 | 0.28 | 0.28 | 0.28 | 0.28 | 0.28 | 0.28 |
| Sulfate | | | | | | | | | | |
| Sodium | 0.800 | 0.800 | 0.800 | 0.800 | 0.800 | 0.800 | 0.800 | 0.800 | 0.800 | 0.800 |
| Chloride** | | | | | | | | | | |
| Sodium Xylenesulfonate** | 0.00 | 0.00 | 0.00 | 0.00 | 0.00 | 0.00 | 0.00 | 0.00 | 0.00 | 0.00 |
| Perfume | 0.700 | 0.750 | 0.700 | 0.350 | 0.700 | 0.750 | 0.700 | 1.000 | 0.650 | 0.730 |
| Sodium Benzoate | 0.250 | 0.250 | 0.250 | 0.250 | 0.250 | 0.250 | 0.250 | 0.250 | 0.250 | 0.250 |
| Kathon | 7.5E-04 | 7.5E-04 | 7.5E-04 | 7.5E-04 | 7.5E-04 | 7.5E-04 | 7.5E-04 | 7.5E-04 | 7.5E-04 | 7.5E-04 |
| Benzyl Alcohol | 0.0225 | 0.0225 | 0.0225 | 0.0225 | 0.0225 | 0.0225 | 0.0225 | 0.0225 | 0.0225 | 0.0225 |
| Water | Q.S. | Q.S. | Q.S. | Q.S. | Q.S. | Q.S. | Q.S. | Q.S. | Q.S. | Q.S. |

*adjustable to achieve target pH
**adjustable to achieve target viscosity
¹Available as N-Hance 3269 from Aqualon
²Available as LR400 from Dow/Amerchol
³Available as Kelcogel from CP Kelco
⁴Available as Methocel from Dow/Amerchol
⁵Available as Klucel from Hercules
⁶Available as Viscasil 330M from GE Silicones
⁷Available as Bruggemann ZC from Bruggemann Chemicals

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Background, Summary of the Invention, and Detailed Description of the Invention are, in relevant part, incorporated herein by reference; the citation of any document is not to be construed as an admission that it is prior art with respect to the present invention.

What is claimed is:

1. A personal care composition comprising:
a. an effective amount of a zinc containing material;
b. a surfactant; and
c. a thickening polymer selected from the group consisting of cationic thickening polymers, nonionic thickening polymers, and mixtures thereof;
wherein said thickening polymer does not bind completely to the zinc ions of said zinc containing material.

2. The personal care composition of claim 1 wherein said zinc containing material has an aqueous solubility within the composition of less than about 25% by weight at 25° C.

3. The personal care composition of claim 1 wherein said surfactant is present in an amount of from about 5% to about 50% of said personal care composition.

4. The personal care composition of claim 1 wherein said thickening polymer is present in an amount of from about 0.01% to about 20% of said personal care composition.

5. The personal care composition of claim 1 wherein the pH of said personal care composition is greater than about 7.

6. The composition according to claim 1 wherein said zinc containing material is selected from the group consisting of inorganic materials, natural zinc containing materials, ores, minerals, organic salts, polymeric salts, or physically adsorbed form material and mixtures thereof.

7. The composition according to claim 6 wherein the inorganic material is selected from the group consisting of zinc aluminate, zinc carbonate, zinc oxide, calamine, zinc phosphate, zinc selenide, zinc sulfide, zinc silicates, zinc silicofluoride, zinc borate, or zinc hydroxide and zinc hydroxy sulfate, zinc-containing layered material, and mixtures thereof.

8. The personal care composition of claim 1 wherein said thickening polymer is a cationic thickening polymer.

9. The personal care composition of claim 8 wherein said cationic thickening polymer is selected from the group consisting of a cationic guar polymer, a Polyquaternium polymer, and mixtures thereof.

10. The personal care composition of claim 1 wherein said thickening polymer is a nonionic thickening polymer.

11. The personal care composition of claim 10 wherein said nonionic thickening polymer is selected from the group consisting of methyl hydroxypropyl cellulose, xanthan gum, alhinate polysaccharide Gellan Gum, polysaccharide gum, hydroxyl propyl cellulose, hydroxyl propyl methyl cellulose, hydroxyl ethyl cellulose, polyalkylene gylcols, and mixtures thereof

12. The personal care composition of claim 10 wherein said nonionic thickening polymer is selected from the group consisting of a nonionic gum polymer, a nonionic hydrophilic cellulose polymer, a nonionic polyethylene glycol polymer and mixtures thereof.

13. The personal care composition of claim 1 further comprising a zinc ionophoric material.

14. The personal care composition of claim 1 wherein said zinc ionophoric material is selected from the group consisting of polyvalent metal salts of pyrithiones, dithiocarbamates, heterocyclic amines, nonsteriodal anti-inflammatory compounds, naturally occurring materials having zinc ionophoric behavior, and derivatives thereof, bio-molecules and peptides, sulfur-based compounds, transport enhancers and mixtures thereof.

15. The personal care composition of claim 14 wherein said zinc ionophoric material is pyrithione or a zinc salt of pyrithione.

16. The personal care composition of claim 1 wherein the viscosity of said personal care composition is from about 15 Pas to about 20,000 Pas at a shear rate of from about 0.0005 s⁻¹ to about 0.005 s⁻¹.

17. The personal care composition of claim 1 wherein the viscosity of said personal care composition is from about 0.001 Pas to about 50 Pas at a shear rate from about 10 s⁻¹ to about 1000 s⁻¹.

18. The personal care composition of claim 1 wherein said personal care composition is a multi-phase personal care composition.

19. A multi-phase personal care composition comprising:
at least two phases wherein at least one phase comprises an effective amount of a zinc containing material, at least one surfactant, and at least one thickening polymer selected from the group consisting of cationic thickening polymers, nonionic thickening polymers, and mixtures thereof;
wherein said thickening polymer does not bind completely to the zinc ions of said zinc containing material; and
wherein said at least two phases are visually distinct phases that are packaged in physical contact and maintain stability.

20. The multi-phase personal care composition according to claim 19 wherein said visually distinct phases form a pattern selected from the group consisting of striped, geometric, marbled, and combinations thereof.

21. The multi-phase personal care composition according to claim 19 wherein at least one phase of said phases is visibly clear.

22. A method of delivering benefits to hair or skin, said method comprising the steps of:
a. topically applying an effective amount of a composition according to claim 1 onto said hair or skin; and
b. removing said composition from said hair or skin by rinsing with water.
